(19) United States
(12) Patent Application Publication
(10) Pub. No.: US 2011/0076240 A1
(43) Pub. Date: Mar. 31, 2011

(54) Antifungal and Anti-Cariogenic Cellobio-Oligosaccharides Produced by Dextransucrase

(76) Inventors: Donal F. Day, Baton Rouge, LA (US); Misook Kim, Baton Rouge, LA (US)

(21) Appl. No.: 12/988,343

(22) PCT Filed: Apr. 20, 2009
(86) PCT No.: PCT/US09/41092

§ 371 Date: Nov. 24, 2010

Related U.S. Application Data
(60) Provisional application No. 61/046,099, filed on Apr. 18, 2008.

(51) Int. Cl. A61K 8/73; A61K 31/702; A61Q 11/00; A61P 31/10; A01P 3/00; A61P 31/04; A61K 31/715; A01N 43/16

(52) U.S. Cl. 424/49; 514/54

(57) ABSTRACT

Cellobio-oligosaccharides (CBO) produced by the dextransucrase-catalyzed transglycosylation reaction of sucrose and cellobiose were discovered to be effective as antifungal agents against dental caries and against fungi who rely on glucan as an integral part of the cell wall, e.g., A. terreus. The cellobio-oligosaccharides were found to be inhibitors of β-(1,3)-glucan synthase, an important enzyme involving in fungal cell wall component synthesis. The CBO caused structural changes in the growing fungal cells. In addition, the CBO were shown to be effective as anti-cariogenic agents in preventing bacterial adherence to teeth by inhibiting the formation of the bacterial plaque (glucans), e.g., that formed by Streptococcus mutans. Cellobio-oligosaccharides produced by dextransucrase were analyzed and shown to have a degree of polymerization (DP) ranging from 3 to 6 glucosyl groups. Examples of these cellobio-oligosaccharides produced by this method include, but are not limited to, trisaccharides such as α-D-glucopyranosyl-(1→2)-β-D-glucopyranosyl-(1→4)-D-glucopyranose and α-D-glucopyranosyl-(1→6)-β-D-glucopyranosyl-(1→4)-D-glucopyranose.

(In countries other than the United States:) The benefit of the Apr. 18, 2008 filing date of U.S. provisional patent application 61/046,099 is Claimed under applicable treaties and conventions. (In the United States:) The benefit of the Apr. 18, 2008 filing date of U.S. provisional patent application 61/046,099 is Claimed under 35 U.S.C. §119(e) in the United States.

This invention was made with the United States government support under contract No. DE-FG36-04GO14236 awarded by the Department of Energy. The United States government has certain rights in this invention.

### TECHNICAL FIELD

This invention relates to compositions and methods using cellobio-oligosaccharides to inhibit caries formation by preventing the production of bacterial biofilms on teeth and to inhibit growth of fungi, e.g., Aspergillus, in part by inhibiting β-1,3 glucan synthase, an enzyme essential for fungal cell wall formation.

### BACKGROUND ART

### Cellobio-Oligosaccharides

Oligosaccharides are carbohydrate polymers, generally with two to ten monomeric residues linked by O-glycosidic bonds. Oligosaccharides have been widely used in food, animal feed, pharmaceutical, and cosmetic industries for a long time due to their beneficial effects on human and animals (Eggleston and Côte, 2003). Most commercial oligosaccharides were originally developed as sweeteners, but currently are valued more as soluble fibers, which decrease gastrointestinal transit time and moderate constipation and diarrhea. Oligosaccharides are considered to be a low calorie food, because they are resistant to attack by digestive enzymes in human and animals and are not absorbed by the host. Oligosaccharides may be produced through microbial fermentation, enzymatic synthesis, or extraction from naturally occurring sources. The major commercial oligosaccharides include cyclomaltodextrins, maltodextrins, fructooligosaccharides, galactooligosaccharides, and soy oligosaccharides (Eggleston and Côte, 2003). Various oligosaccharides have been investigated for physiological functions including immune-stimulating, anti-cariogenic, and prebiotic compounds as well as sweeteners, stabilizers, and bulking agents (Eggleston and Côte, 2003; Otaka, 2006). Most of the beneficial effects related to health are reported to be as inhibitors against enzymes involved in carbohydrate metabolism.

Enzymatic production of oligosaccharides has many advantages over other methods, especially chemical methods. In most cases, the enzymatic synthesis of oligosaccharides has used transglycosylation reactions between a specific donor and a relatively large variety of structurally different acceptors. The configuration of the glycosidic bond produced is a function of the specificity of the transfer by the specific enzyme (Fu et al., 1990; Robyt, 1995). Various oligosaccharides have been produced by enzymatic transfer reactions from glucansucrases, amylosucrases, cyclodextrin glucanosyltransferases, and sialy transferases.

Dextransucrase (EC 2.4.5.1) is a glucosyltransferase, which catalyses the transfer of D-glucopyranosyl residues from sucrose to dextran. It catalyzes the synthesis of a dextran containing 50% or more α-1,6 glucosidic bonds in the main chain. However, in the presence of an alternate efficient acceptor molecule, its action changes to produce oligosaccharides.

Cellobiose is a disaccharide composed of two glucose molecules linked with a β-1,4 bond, which is produced during the enzymatic hydrolysis of cellulose. Another class of sugars containing cellobiose as a component are produced by transglycosylation reactions (Lee et al., 2003; Morales et al., 2001). Acarbose analogues containing cellobiose were prepared by the reaction of acarbose and cellobiose with Bacillus stearothermophilus alpha maltogenic amylase. Cellobiose-acarbose analogues inhibited β-glucosidase, whereas acarbose did not. Oligosaccharides with branched chains, using cellobiose as acceptor, were produced in a reaction catalyzed by alternansucrase from Leuconostoc mesenteroides NRRL B-23192 (Morales et al. (2001). Leuconostoc mesenteroides B512 FMCM produces an extracellular dextransucrase which synthesizes a dextran that has 95% α-(1→6) linear and 5% α-(1→3) branched linkages, and can transfer glucosyl units from sucrose onto an acceptor to produce oligosaccharides (Lindberg and Svensson, 1968.).

L. mesenteroides B-512 F dextransucrase synthesized α-D-(1→2)-glucopyranosyl cellobiose and α-D-(1→6)-glucopyranosyl cellobiose in the presence of cellobiose. (Morales et al., 2001). Besides these two products, α-D-glucopyranosyl-(1→3)-α-D-glucopyranosyl-(1→6)-α-D-glucopyranosyl-(1→6)-cellobiose and α-D-glucopyranosyl-(1→6)-α-D-glucopyranosyl-(1→6)-cellobiose were synthesized when cellobiose is used as the acceptor molecule from sucrose using alternansucrase from L. mesenterodies B-23192. However, the dextransucrase from L. mesenterodies B-512 F is known primarily to transfer the D-glucose residue from sucrose to the non-reducing end 6-hydroxyl group of mono- and higher-saccharides in the presence of an acceptor molecule (Robyt, 1995; Robyt and Eklund, 1983). The types of oligosaccharides synthesized by L. mesenteroides B-512 F dextransucrase apparently depends on the type of acceptor molecule. In the presence of β-glucosidic linkages in the acceptor molecule, the specificity of dextransucrase is changed to transfer the 2-OH group at the reducing end glucose rather than transfer 6-OH at the non-reducing end (Yoon and Robyt, 2002; Robyt, 1995; Robyt and Eklund, 1983). For example, dextransucrase transfers the D-glucose residue to the non-reducing end OH of maltose or isomaltose in the presence of maltose or isomaltose and transfers the D-glucose from sucrose to the reducing end D-glucose as well as the 6-OH groups of the non-reducing end in the presence of maltotriose and maltotetraose (Fu and Robyt, 1990). When the acceptor molecule is lactose or raffinose, L. mesenteroides B-512 F dextransucrase transfers D-glucose from sucrose to the OH group at C-2 of the D-glucose residue (Robyt, 1995; Robyt and Eklund, 1983).

Oligosaccharides and Cariogenicity

Dental caries is decay of the teeth of mammals which is mainly caused by oral bacteria such as Streptococcus mutans and S. sobrinus. S. mutans and S. sobrinus synthesize extracellular, water-insoluble glucans from sucrose by glucosyl transferases (Hamada and Slade, 1980). The insoluble glucans become plaque on teeth and result in tooth decay. Tooth decay is promoted both by the additional bacteria that adhere to the tooth due to the glucans, but also by the increase in acid. S. mutans and S. sobrinus synthesize intracellular polysaccharides as carbohydrate reserves, which can be converted to acids when dietary carbohydrates are available (Marsh, 1999). One of the glucosyl transferase of these oral streptococci bacteria is mutansucrase, which produces glucans with a highly branched structure from sucrose and a majority of α-(1-3)-glucosidic linkages. The glucan compound produced by mutansucrase is called mutan, α-(1-3)-_{D}-Glucan. Mutan is a water-insoluble adherent, and enhances the attachment of bacteria to tooth surfaces. Maltooligosylsucrose (commonly known as Coupling sugar) and palatinose (also known as isomalturose) have been reported as preventing the occurrence of dental caries (Otaka, 2006).

Oligosaccarides as Antifungal Agents

Antifungal agents are compounds that selectively eliminate fungal pathogens from a host, with minimal toxicity to the host. Control of fungi is crucial to prevent losses in food supplies and to decrease the fatal effects of fungal infections in patients with weakened immune systems. Studies of antifungal agents have lagged behind research on antibacterial agents. Bacteria are prokaryotic and offer numerous structural and metabolic targets that differ from those in human hosts. However, fungi are eukaryotic with the same biochemistry as mammalian cells resulting in many similarities between fungi and host cells in both cell structure and metabolism. Due to these similarities, many antifungal agents can be toxic to host cells as well as fungi. This causes toxic side effects on exposure to antifungal agents. There is a lack of selective toxicity of antifungal agents producing a poor selection of clinically available drugs. In addition, poor solubility of many antifungal agents and poor absorption through the gastrointestinal tract reduce feasibility of oral administration and increase the levels of toxicity associated with the use of antifungal agents.

Fungal infections in humans can cause a high rate of fatalities (50-90%). Opportunistic infectious fungal diseases have emerged as a major cause of morbidity and mortality in immuno-compromised people. Aspergillus fumigates, Aspergillus flavus, and Aspergillus terreus are the three fungal species which cause about 95% of the pathogenic cases in humans. Infection with A. terreus is a growing concern because the infection is more aggressive and has a higher mortality rate than infections caused by other Aspergillus species. Aspergillosis is a unique disease because the fungus enters the body as conidia and then grows as a mycelium. Aspergillus can cause blockage of blood vessels, inflammation of the inner lining of the heart, clots in the heart vessels, and serious impairment of lung function. Unfortunately, A. terreus is usually resistant to antifungal agents, including amphotericin B. Therefore, there is a real need for new effective antifungal compounds, especially against A. terreus.

Antifungal agents are classified into three groups: (1) antimicrobial agents affecting fungal sterol (e.g., fluconazole, itraconazole, ketoconazole, miconasol, allymine, amphotericin B, nystatin, and flucytosine), (2) agents inhibiting nucleic acids (e.g., 5-fluorocytosine), and (3) agents active against fungal cell walls (e.g., nikkomycin, demethylallosamidin, and polyxin). Glucan synthesis inhibitors are compounds active against fungal cell walls. Fungal cell walls are composed of β-(1,6)-glucan, mannan, or mannoprotein in the outer layers; and β-(1,3)-glucan and chitin in the inner layers. Examples of fungi genera known to have glucan synthase include Candida, Aphanomyces, Paracoccidioides, Saprolegma, Aspergillus and Cordyceps. So far, most glucan synthase inhibitors have been categorized into three chemical classes of compounds: (1) lipopeptides comprising cyclic hexapeptides N-linked to a fatty acyl side chain, (2) papulacandins consisting of a modified disaccharide linked to two fatty acyl chains; and (3) acidic terpenoids (Douglas, 2001; Onishi et al., 2000; Tracz 1992; Traxler et al., 1977). Most glucan synthase inhibitors induce profound morphological changes in fungal hyphae which have been correlated with inhibition of glucan synthase (Kurtz et al., 1994; Bozzola et al., 1984; Cassone et al., 1981).

Inhibitors of β-(1,3)-glucan synthesis are a new therapeutic class for treating serious fungal infection. The (1,3)-β-D-glucan synthase inhibitors are an effective treatment for fungal infections because these agents inhibit fungal cell wall synthesis, a target unique to lower eukaryotes (Onishi et al, 2000).

Echinocandins, pneumocandins, and papulacandins are known inhibitors of β-(1,3)-glucan synthase. However, they have complicated structures and are hydrophobic in nature. Although the abundance of 1,3-β-_{D}-glucans in the cell walls formed during different stages of the A. fumigatus life cycle is not well characterized, the focus of new cell wall synthesis is the hyphae during vegetative growth (Archer, 1977; Beauvais et al., 2001: Ruiz-Herrera, 1992). Inhibition of 1,3-β-D-glucan synthesis has profound effects on cell wall structure in A. fumigatus (Kurtz et al., 1994). Inhibition of glucan synthesis results in structural changes, characterized as pseudohyphae, swollen hyphae, thickened cell wall, or buds failing to separate from mother cells (Kurtz et al., 1994; Bozzola et al., 1984; Cassone et al., 1981). Pneumocandin-treated A. fumigates caused swelling and distension of the hyphae (Kurtz et al, 1994).

A small and simple sugar acid, _{D}-gluconic acid from Pseudomonas strain AN5, has been reported to have antifungal activities against the take-all disease of wheat caused by Gaeumannomyces graminis var. tritici (Kaur et al., 2006). Some researchers have reported that cellobiose-based lipids have fungicidal activities. Complex cellobiose-lipids of yeast fungi Cryptococcus humicola and Pseudozyma fusiformata (ustilagic acid B) inhibited the growth of a number of species, important for medicine: Candida. albicans, C. glabrata, C. viswanathii, F. neoformans, and Clavispora lusitaninae (Kulakovskaya et al., 2007; Kulakovskaya et al., 2006). These lipids may stimulate the release of ATP from the test culture cells, indicating an increase in the permeability of plasma membrane, and resulting in cell death (Puchkov et al., 2001; Kulakovskaya et al., 2004). Mimee et al. (2005) isolated flocculosin, a low molecular weight cellobiose-lipid, from the yeast-like fungus Pseudozyma flocculosa to investigate antifungal activity. Flocculosin significantly inhibited the growth of Candida lustitaniae, C. neoformans, Trichosporon asahii, and C. albicans. Synergistic activity was also verified between flocculosin and amphotericin B. Most isolated cellobiose-lipids have considerable efficacy as potential antifungal agents under the acidic condition (Kulakovskaya et al., 2007; Mimee et al., 2005).

A cellotriose, comprising three glucose molecules linked with only β-1,4, enhanced glucan synthase activity isolated from Euglena gracilis (Marechal and Goldemberg, 1964). Cellobiose has been reported to be a stimulator for glucan synthase production in sugar beets (Morrow and Lucas, 1986) and in Euglena gracilis (Marechal and Goldemberg, 1964). However, cellobiose was not found to stimulate the production of glucan synthase in S. cerevisiae (Lopez-Romero and Ruiz-Herrera, 1978) or the germinating peanut, Arachis hypogaea (Kamat et al., 1992). A very simple, sugar-based chemical, δ-gluconolactone, was an effective inhibitor of (1→3)-β-_{D}-glucan synthase in the sugar beet (Morrow and Lucas, 1987) and in S. cerevisiae (Lopez-Romero and Ruiz-Herrera, 1978).

### DISCLOSURE OF INVENTION

We have discovered that cellobio-oligosaccharides (CBO) produced by the dextransucrase-catalyzed transglycosylation reaction of sucrose and cellobiose are effective as antifungal agents and against bacterial-caused dental caries. The cellobio-oligosaccharides were found to be inhibitors of β-(1,3)-β-D-glucan synthase, an important enzyme involved in synthesis of the fungal cell wall, resulting in structural changes in the growing fungal cells. In addition, these CBO were also shown to be effective as anti-cariogenic agents by preventing bacterial adherence to teeth because the CBO prevent the production of insoluble, adherent glucans, e.g., mutan. Cellobio-oligosaccharides produced by dextransucrase were analyzed and shown to have a degree of polymerization (DP) ranging from 3 to 6 glucosyl groups. Examples of these cellobio-oligosaccharides produced by this method include trisaccharides such as α-D-glucopyranosyl-(1→2)-β-D-glucopyranosyl-(1→4)-D-glucopyranose and α-D-glucopyranosyl-(1→6)-β-D-glucopyranosyl-(1→4)-D-glucopyranose, and smaller amounts of tetrasaccharides, pentasaccharides, and hexasaccharides.

### BRIEF DESCRIPTION OF THE DRAWINGS

FIG. 1 illustrates the results of high performance anion exchange chromatography (HPAEC) (left) and thin layer chromatography (TLC) (right) of the cellobio-saccharides produced by dextransucrase-catalyzed transglycosylation of sucrose and cellobiose, with A and B representing trisaccharides, C representing a tetrasaccharide, D representing a pentasaccharide, and E representing a hexasaccharide.

FIGS. 2A and 2B illustrate the proposed chemical structures of two trisaccharides: α-D-glucopyranosyl-(1→2)-β-D-glucopyranosyl-(1→4)-D-glucopyranose (FIG. 2A) and α-D-glucopyranosyl-(1→6)-β-D-glucopyranosyl-(1→4)-D-glucopyranose (FIG. 2B).

FIG. 3 illustrates the amount of insoluble glucan formed by bacterial mutansucrase expressed as a percent formed in the control condition under the following conditions: Control; addition of cellobiose (Cellobiose); and addition of cellobio-oligosaccharides (CBO), with bars representing the standard deviation of the mean.

FIG. 4 illustrates the amount of insoluble glucan on the walls of glass vials under different experimental conditions: Control; addition of 50 mM cellobiose (Cellobiose); and addition of 50 mM cellobio-oligosaccharides (CBO).

FIG. 5 illustrates the (1→3)-β-D-glucan synthase activity expressed as a percent of control activity in the presence of increasing concentrations of cellobio-oligosaccharides (0.12 g/ml; 0.24 g/ml; 0.36 g/ml; and 0.48 g/ml.

FIG. 6 illustrates a Lineweaver-Burk plot showing the effect on the activity of (1→3)-β-D-glucan synthase of the addition of increasing concentrations of cellobio-oligosaccharides (0 g/ml, 0.24 g/ml and 0.36 g/ml).

FIGS. 7A and 7B depict the results of Scanning Electron Microscopy (SEM) on A. terreus cells under two experimental conditions: FIG. 7A, cells grown without cellobio-oligosaccharide (arrows show sporulation); and FIG. 7B, cells grown with 50 mM cellobio-oligosaccharides.

FIGS. 8A-8D illustrate the in vitro growth of A. terreus in potato dextrose media in both broth (in tubes; FIGS. 8A and 8B) and agar (in petri dishes; FIGS. 8C and 8D) without cellobio-oligosaccharides (FIGS. 8A and 8C) and with 50 mM cellobio-oligosaccharides (FIGS. 8B and 8D):

### MODES FOR CARRYING OUT THE INVENTION

Cellobio-oligosaccharides (CBO) as described herein are produced by the dextransucrase-catalyzed transfer of one or more D-glucosyl moieties from sucrose to cellobiose. They are suitable for use in the inhibition of fungal growth, e.g., A. terreus, and the inhibition of dental caries. During the production of CBOs, the glucosyl units are added through an α(1→6) or α(1→2) linkage onto cellobiose. The mixture of oligosaccharides having degrees of polymerization from 3 to 6 glucosyl groups is produced by the dextransucrase catalyzed reaction using sucrose and cellobiose. The size of oligosaccharides (DP) depends on the number of glucosyl units attached onto the cellobiose molecule (two glucosyl units of the original cellobiose molecule). For example, a single glucosyl unit attached to the cellobiose would be a trisaccharide with a DP of 3. The majority of cellobio-oligosaccharides produced were trisaccharides. An example of cellobio-oligosaccharides useful as antifungal and anticariogenic agents include a mixture composed of about 66% trisaccharides with two different structures, about 13% tetrasaccharides, about 13% pentasaccharides, and about 8% hexasaccharides.

Cellobio-oligosaccharides are simple molecules and water soluble, thus easy for use. As with other oligosaccharides, they are expected to be non-toxic to humans. The cellobio-saccharides could be used with other antifungal agents or with other anti-cariogenic agents.

### Example 1

### Production and Characterization of the Produced Cellobio-Oligosaccharides

Production of Dextransucrase: Leuconostoc mesenteroides B-512 FMCM, a constitutive mutant from the parent B512 F for dextransucrase production, was obtained from Dr. Doman Kim (Chonnam National University, Gwangju, South Korea; and as described in Kim and Kim, 1999). The culture was grown at 30° C. in LM medium [0.5% (w/v) yeast extract, 0.5% (w/v) peptone, 2% (w/v) K₂HPO₄, 0.02% (w/v) MgSO₄.7H₂O, 0.001% (w/v) NaCl, 0.001% (w/v) FeSO₄.7H₂O, 0.001% (w/v) MnSO₄.H₂O, 0.013% (w/v) CaCl₂.2H₂O] containing 2% glucose or 2% sucrose. The culture could also be maintained on glucose-LM medium containing 2% glucose and 1.5% agar at 4° C., and transferred biweekly. For growth measurement, samples of 5 ml were taken at various intervals for 48 h. Bacterial growth was measured at 660 nm in a spectrophotometer using a 1 cm optical cuvette, and pH was measured directly. The source of the chemicals and other agents used in the following examples were common commercial sources, usually Sigma Co. (St. Louis, Mo.), unless otherwise indicated.

Leuconostoc mesenteroides B-512 FMCM was sub-cultured by three successive transfers including 1 ml sucrose-LM and glucose-LM medium, 40 ml, and 1 L glucose-LM media to build sufficient volume for inoculation of the final fermentation. The inoculums were 2-5% (v/v) with cultures grown for 16 h at 30° C. with shaking at 150 rpm. For dextransucrase production, a 400 ml culture was inoculated to 14 L of LM medium containing 2% glucose and incubated for 48 h at 30° C. The pH and agitation were not controlled during fermentation. After harvesting, cells were removed by centrifugation at 6,000 rpm×g for 30 min. The cell-free culture was concentrated 10-fold using membrane filtration (100K cutoff) and washed with 2 volumes of 20 mM sodium citrate buffer, pH 5.2. Tween 80 and NaN₃ were added at concentrations of 1 mg/ml and 0.2 mg/ml to enzyme solution.

Dextransucrase activity was determined by incubating the enzyme with 100 mM sucrose in 20 mM sodium citrate buffer, pH 5.2 for 1 h at 30° C. and then boiling for 5 min to terminate the enzyme reaction. One unit of dextransucrase activity was defined as that amount of enzyme releasing 1 μM fructose per min from 100 mM sucrose. The fructose was determined by high performance liquid chromatography (HPLC) using an Aminex HPX 87K column (300 mm×7.8 mm) and a HPLC analyzer coupled to a refractive index detector. The column was maintained at 85° C. and 0.01 M K₂SO₄ was used as a mobile phase at a flow rate 0.6 ml/min.

Transglycosylation using dextransucrase: Transglycosylation reactions were performed in 500 ml of 20 mM citrate buffer (pH 5.2) including 300 mM of sucrose, 250 mM of cellobiose, and 54 U dextransucrase at 30° C. with shaking at 150 rpm. The reaction was performed until the sucrose was depleted and then terminated by heating for 20 min at 95° C. A reaction product was centrifuged at 6,500 rpm for 45 min for the removal of insoluble polysaccharide. The soluble polysaccharide was precipitated with an equal volume of ethanol which was slowly added to the supernatant and the resulting solution stored in a refrigerator for 2 h. The precipitate was eliminated by centrifugation at 6,500 rpm for 45 min.

Initial Characterization of Produced Cellobio-oligosaccharides: The supernatant from above was analyzed by using thin layer chromatography (TLC). The TLC samples were loaded onto a Whatman K5 silica gel plate. The plate was irrigated three times with 2:5:1.5 volume parts of nitromethane-1-propanol-water. The carbohydrates on the TLC plate were visualized by dipping the plate into a methanol solution containing 0.3% (w/v) N-(1-naphthyl)ethylenediamine and 5% (v/v) sulfuric acid, followed by heating at 110° C. for 15 minutes. The relative percent of carbohydrates was determined using Scion image analyzer software.

Oligosaccharides were also analyzed by high performance anion exchange chromatography (HPAEC) using a Dionex Carbo-Pac PA 100 column (250×4 mm) by gradient elution using 1 M NaOH, water and 480 mM sodium acetate at a constant flow rate of 0.5 mL/min. Oligosaccharide detection was carried out with an electrochemical detector (ED 40). The supernatant was concentrated 10-fold using a Rotary evaporator and then freeze dried.

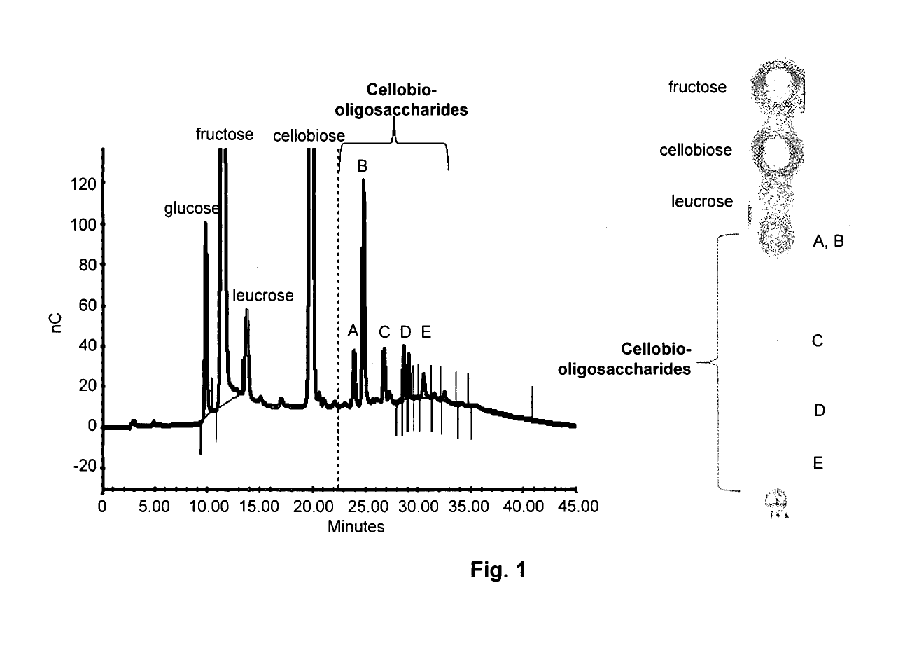

As shown in FIG. 1, the transglycosylation reaction between cellobiose and sucrose by L. mesenteroides B-512 FMCM dextransucrase produced one major product (B), several minor products (A, C, D, and E), fructose and leucrose (FIG. 1). The concentrations of each peak were 1.5 mg/mL for peak A, 5.5 mg/mL for peak B, 1.4 mg/mL for peak C, 1.4 mg/mL for peak D, and 0.9 mg/mL for peak E (See also, Table 1).

Purification: The crude oligosaccharide supernatant was loaded onto a Bio-Gel P2 (fine) column (1.5 cm×115 cm), and eluted with water, and the elution collected in 0.5-1.0 ml fractions. The purity of each fraction was determined using either TLC or HPAEC as described above. Those fractions with the same degrees of polymerization were pooled and freeze dried for structure analysis. For the anti-cariogenic and antifungal experiments, the fractions containing CBOs were recombined making a mixture that was representative of what was initially formed and was composed of about 66% trisaccharides with two different structures, about 13% tetrasaccharides, about 13% pentasaccharides, and about 8% hexasaccharides. This mixture thus contained primarily trisaccharides.

Mass Spectrometry Analysis: Mass spectrometry data of the purified oligosaccharides were obtained from electrospray (MS-ES) measurements. The solvent was ultrapure water at 7 μl/min and detection was performed in the positive mode. The mass of the product for peaks A and B indicated 504.07 g/mol, for peak C 660.02 g/mol, for peak D 828.28, and for peak E 990.33 (Table 1). The masses of these reaction products increased over that of cellobiose by a single D-glucose residue (M.W. 162 g/mol). Therefore, peaks A and B represented compounds that were trisaccharides, peak C represented a tetrasaccharide, peak D represented a pentasaccharide, and peak E represented a hexasaccharide.

TABLE 1
Concentration, apparent yield, molecular
mass of cellobio-oligosaccharides

| | Concentration | Apparent | Molecular |
|---|---|---|---|
| Compound | (mg/mL) | yield (%) | Mass (g/mol) |
| A | 1.5 | 14.0 | 504.07 |
| B | 5.5 | 52.7 | 504.07 |
| C | 1.4 | 12.9 | 660.02 |
| D | 1.4 | 13.3 | 828.28 |
| E | 0.9 | 8.1 | 990.33 |

About 50 mg from the purified oligosaccharides were exchanged three to four times with 600 μl pure D₂O and lyophilized twice and then dissolved in 600 μl pure D₂O, and placed into 5 mm NMR tubes. NMR spectra were produced using a spectrometer, operating at 500 MHz for H and 125 MHz for ¹³C at 25° C. It was examined for the linkages between cellobiose and glucose from homonuclear correlation spectroscopy (COSY), total correlation spectroscopy (TOCSY), rotating frame overhause effect spectroscopy (ROESY), heteronuclear single quantum coherence (HSQC), and heteronuclear multiple quantum coherence spectroscopy (HMQC) spectra.

Structural Analysis Using NMR: The structures of the major transglycosylation products corresponding to peaks A and B were determined by ¹H and ¹³C nuclear magnetic resonance (NMR) spectrometry to determine the synthetic modes of cellobio-oligosaccharides. The proton signals were assigned from analyses of ¹H/¹H-COSY and ¹H/¹H-TOCSY spectra. After the assignment of all proton signals, the corresponding ¹³C resonances were allowed by ¹H/¹³C-HSQC spectrum, followed by ROESY and HMQC. All assignments of the cellobio-oligosaccharides are shown in Table 2.

The NMR assignments indicated two forms of trisaccharides. The smaller amount of trisaccharide was noted as product A and the larger amount of trisaccharide as product B. When the integral of III-1 proton at 5.06 ppm was determined, the total integral of two III-1 protons at 5.33 and 4.96 ppm were 2.2 (Data not shown). Therefore, a trisaccharide having III H-1 at 5.06 ppm was determined as a product A and the other as a product B.

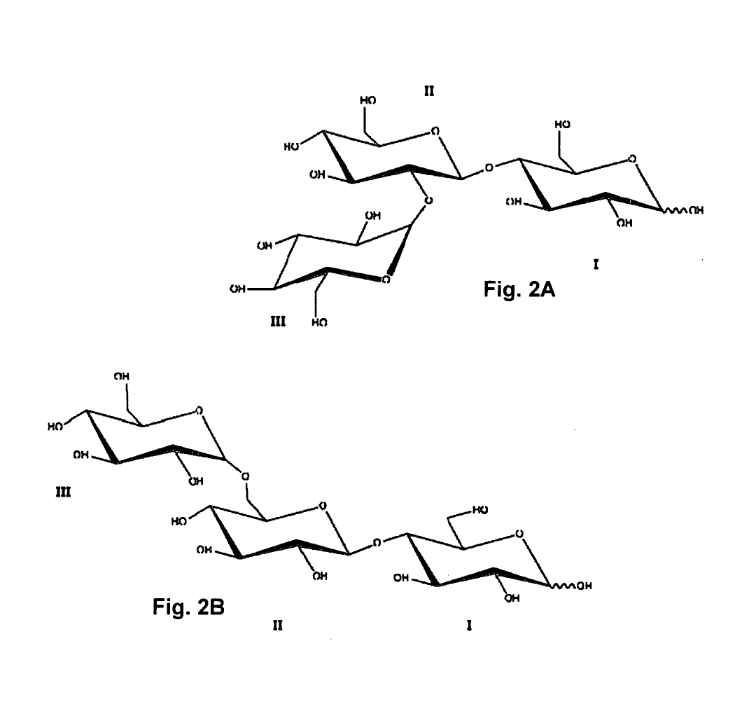

The new anomeric proton signals at 5.06 ppm (J=3.5 Hz, doublet signal) was assigned, indicating that a glucosyl residue was connected to cellobiose with α-linkage. In product A, the ¹³C-chemical shift in cellobiose before and after the addition of α-D-glucopyranose to cellobiose for C-6 was changed from 60.94 ppm to 66.35 ppm (Table 2). This chemical shift change is characteristic of the attachment of a D-glucopyranose unit to the original glucoside or aglycone. Except for this change for C-6, the spectra of product A gave no resonance changes. Therefore, the NMR result indicates that the D-glucopyranose unit was attached to the cellobiose ring by an α-(1→6) linkage. This cellobio-oligosaccharide structure is shown in FIG. 2B.

In product B, the ¹H chemical shifts of the new anomeric carbon (C-1) were 5.33 and 4.96 ppm with a coupling constant of 3.5 Hz, indicating that they were α-conformation (Table 2). The corresponding ¹³C chemical shifts appeared at 98.29 and 98.59 ppm (Table 2). The ¹³C chemical shift of C-1 in α-D-glucopyranose-(1→2)-β-D-glucopyranose was 98.6 ppm, which indicates an α-(1→2) linkage (Bock et al., 1986). Evidence for this linkage was supported by a downfield ¹³C shift for II C-2 of cellobiose from 73.54 to 76.73 (Table 2). These results identified the cellobio-oligosaccharide structure as α-D-glucopyranosyl-(1→2)-β-D-glucopyranosyl-(1→4)-D-glucopyranose (FIG. 2A).

TABLE 2
¹H NMR and ¹³C NMR chemical shifts^{a} for product A and B produced by the
reaction of dextransucrase with sucrose and cellobiose (units: ppm).

| | | | Cellobiose (δ) | CBO-A^{b} (δ_{A}) | | | CBO-B^{c} (δ_{B}) | | |
|---|---|---|---|---|---|---|---|---|---|
| | | | δ_{C} | δ_{AC} | δ_{AC} − δ_{C} | δ_{H} | δ_{BC} | δ_{BC} − δ_{C} | δ_{H} |
| I^{d} | α-Glc | 1^{e} | 92.20 | 92.33 | 0.13 | 5.19 | 89.70 | −2.51 | 5.41 |
| | | 2 | 71.60 | 71.83 | 0.24 | 3.56 | 70.33 | −1.27 | 3.65 |
| | | 3 | 71.71 | 72.28 | 0.58 | 3.94 | 72.24 | 0.53 | 3.94 |
| | | 4 | 79.11 | 79.03 | −0.07 | 3.68 | 79.06 | −0.05 | 3.68 |
| | | 5 | 70.48 | 70.40 | −0.08 | 3.89 | 70.40 | −0.08 | 3.89 |
| | | 6 | 60.28 | 60.63 | 0.35 | 3.80 | 60.28 | 0.00 | 3.90 |
| | β-Glc | 1 | 96.12 | 96.54 | 0.41 | 4.78 | 96.31 | 0.18 | 4.63 |
| | | 2 | 74.26 | 73.58 | −0.68 | 3.39 | 73.74 | −0.51 | 3.27 |
| | | 3 | 74.66 | 73.46 | −1.20 | 3.70 | 73.82 | −0.84 | 3.49 |
| | | 4 | 78.97 | 79.07 | 0.10 | 3.69 | 79.87 | 0.90 | 3.61 |
| | | 5 | 75.16 | 73.58 | −1.59 | 3.67 | 73.79 | −1.37 | 3.47 |
| | | 6 | 60.42 | 61.232 | 0.81 | 3.89 | 61.13 | 0.71 | 3.72 |
| II | β-Glc | 1 | 102.93 | 103.46 | 0.53 | 4.50 | 102.84 | −0.09 | 4.48 |
| | | 2 | 73.54 | 73.30 | −0.24 | 3.30 | 76.73 | 3.20 | 3.30 |
| | | 3 | 76.86 | 76.12 | −0.74 | 3.49 | 76.12 | −0.74 | 3.49 |
| | | 4 | 69.82 | 70.11 | 0.28 | 3.37 | 69.72 | −0.10 | 3.38 |
| | | 5 | 76.34 | 75.32 | −1.02 | 3.62 | 75.10 | −1.24 | 3.71 |
| | | 6 | 60.94 | 66.35 | 5.42 | 3.88 | 61.28 | 0.34 | 3.68 |
| III | α-Glc | 1 | | 96.89 | | 5.06 | 98.30/98.59 | | 5.33/4.96 |
| | | 2 | | 71.74 | | 3.53 | 71.96 | | 3.53/3.55 |
| | | 3 | | 73.37 | | 3.77 | 73.37 | | 3.77 |
| | | 4 | | 69.82 | | 3.43 | 69.82 | | 3.43 |
| | | 5 | | 70.52 | | 3.95 | 70.52 | | 3.95 |
| | | 6 | | 61.31 | | 3.78 | 61.31 | | 3.78 |

^{a}Chemical shifts were measured at 125 MHz for ¹³C NMR and 500 MHz for ¹H NMR in D₂O
at 25° C. with acetone as an internal standard.
^{b}α-D-glucopyranosyl-(1→6)-cellobiose.
^{c}α-D-glucopyranosyl-(1→2)-cellobiose.
^{d}Each of the residues of cellobio-oligosaccharide is designed by Roman Numerals, started with I at the reducing-end residue.
^{e}The position of carbon and proton and the number starts from the anomeric carbon in a residue.

### Example 2

### Cellobio-Oligosaccharides as Inhibitors of Cariogenicity

Isolation of Oral Bacteria and Production of Mutansucrase: Oral bacteria were collected by a cotton swab from human teeth and streaked onto a brain heart infusion (BHI) agar containing 4% sucrose. The culture was grown at 37° C. until visible colonies of Streptococcus mutans and S. sorbrinus appeared. The colonies were grown in 1 L BHI at 37° C. with shaking at 150 rpm for 24-36 h to induce production of mutansucrase secreted outside the cell membrane. After incubation, the culture was harvested by centrifugation, and the supernatant with the mutansucrase was concentrated to 100 ml using a 30 K cut-off membrane filter. One unit of mutansucrase in the concentrated supernatant was defined as the amount of enzyme that catalyzes the formation of 1 μmol of fructose per minute at 37° C., pH 7.0, from 100 mM sucrose.

Inhibition of Insoluble Glucan Synthesis: The inhibition of CBO on the synthesis of water insoluble glucans by oral Streptococcus species was determined. In the following experiments, CBO is the mixture of cellobio-oligosaccharides as described above in Example 1 which contains a majority of trisaccharides (>60%). Streptococcus species were inoculated in 2×BHI broth containing 1M sucrose for three treatments: one control (no additions), one with 50 mM CBO (primarily trisaccharides) and one with 50 mM cellobiose. These cultures were grown in glass vials at 37° C. for 48 h.

Inhibition of Mutansucrase: Mutansucrase in the concentrated supernatant from above was incubated in 20 mM HEPES (pH 7.0) containing 1 M sucrose in glass vials at 37° C. for 48 h under two conditions: a control (no additions) and with addition of 50 mM CBO. The supernatants of individual reaction mixtures were discarded such that the insoluble glucans remained in the vial. To compare the amount of insoluble glucans produced, the synthesized glucans on the glass were washed with a HEPES buffer and dissolved in 0.5 N NaOH. The resulting wash was used to measure the soluble glucans. The absorbance of water insoluble glucans was measured at 550 nm. For visualization, synthesized glucans were dyed with a drop of dental disclosing solution.

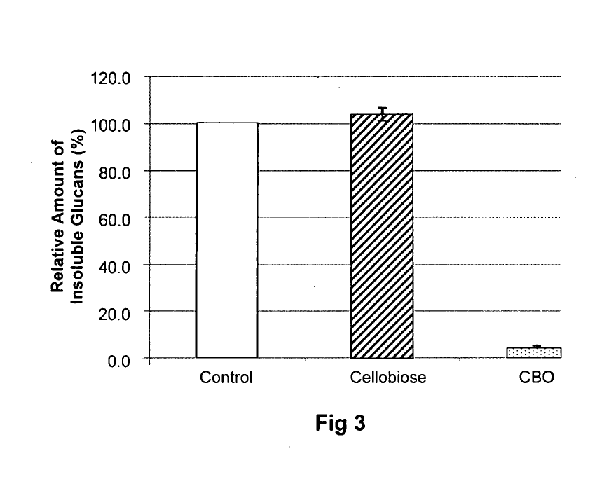

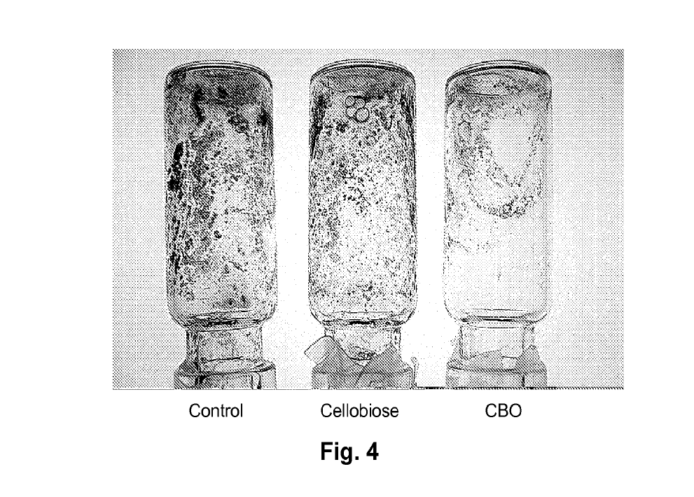

The CBO effectively inhibited the synthesis of water insoluble glucans in the presence of sucrose (FIG. 3). In the presence of 50 mM CBO and 1 M sucrose, only 4% of insoluble glucans were produced when compared to that produced with the control mixture. Addition of cellobiose (50 mM) did not affect the insoluble glucan formation as measured using absorbance at 550 nm. (FIG. 3) Insoluble glucans in solution were swirled along the inner layer of a glass vial, and the liquid was discarded. Then, the carbohydrates were dyed with a dental disclosing solution. The insoluble glucans adhered to an inner layer on a glass vial as dental plaque does on teeth (FIG. 4). The quantity of insoluble glucans that adhered to glass was less for the CBO mixture than the control or the cellobiose treatment. Without wishing to be bound by this theory, it is believed that the inhibitory effect of CBO was primarily the inhibition of the synthesis of glucan by mutansucrase by an acceptor reaction of glucosyltransferase, leading to termination of glucan synthesis from sucrose. Thus CBOs can be effective anti-caries ingredients in dental care products, such as toothpaste or mouth wash.

### Example 3

### Cellobio-Oligosaccharides as Inhibitors of Fungal Growth

Collection and Growth of Aspergillus: A. terreus, ATCC No. 20514 (American Type Culture Collection, Manassas, Va.), was maintained on potato dextrose agar (PDA) medium for 5 to 7 days at 28° C. Conidia were collected with a cotton swab and suspended in 0.9% NaCl solution with 0.05% Tween 20. The heavy particles were allowed to settle for 2 h in cold solution. The viability was confirmed by plating serial dilutions onto PDA plates. For determination of the morphological changes, 2.5×10⁴ conidia were inoculated into 2.9 ml of potato dextrose broth (PDB) with CBO and incubated for 10 days at 28° C. In this experiment and those following, CBO refers to the mixture of cellobio-oligosaccharides as described above in Example 1 which contains a majority of trisaccharides (>60%).

Inhibition of (1,3)-β-_{D}-Glucan Synthase: For glucan synthase production, 4.5×10⁸ conidia were inoculated into 500 ml of YME medium (0.4% yeast extract, 1.0% malt extract, and 0.4% dextrose), and incubated at room temperature for 1 to 2 days with shaking at 150 rpm. The spherical mycelia grown on YME medium with shaking were harvested by centrifugation at 1,500×g for 10 min. Cells were washed extensively with water and then centrifuged at 1,500×g for 10 min. Cell breakage was performed using 20 cycles (1 min each) of vortexing with pre-chilled glass beads in chilled extraction buffer containing 50 mM N-2-hydroxyethylpiperazine-N′-2-ethanesulfonic acid (HEPES; pH 7.2), 1 mM EDTA, 1 mM dithiothreitol (DTT), 10% glycerol, 1 μg of leupeptin per ml, and 10 μM GTPrS at approximately 5 ml of buffer per cell. Cells were cooled for 5 min on ice between cycles. The homogenate was centrifuged at 1,500×g for 10 min to remove cell debris. After centrifugation at 23,000×g for 10 min to remove mitochondrial membranes, the supernatant was ultra-centrifuged at 100,000×g for 1 hr to recover microsomal membranes in a pellet. This pellet was resuspended in one-tenth the original volume of cold storage buffer containing 50 mM HEPES (pH 7.2), 1 mM EDTA, 1 mM DTT, and 20% glycerol. Protein concentration of isolated (1,3)-β-glucan synthase was 2.5 mg/ml. All procedures for enzyme preparation were carried out at 4° C. Protein concentration was determined by the Bio-Rad Protein Assay (Bio-Rad Laboratories, Hercules, Calif.) with bovine serum albumin as standard.

Glucan synthase (GS) activity was determined by the modification of a fluorescence method, as described in Shedletzky et al. (1997). The assay mixture (150 μl) contained 27 mM HEPES (pH 7.2), 7 μM GTP, 1.3 mM EDTA, 0.17% Brij 35, 2.2% glycerol, 0.7 mM UDP-Glc, and isolated GS enzyme (0.83 μg/μl). For inhibition studies, CBO at concentrations from about 0.12 to about 0.63 μg was added to the desired mixture. Reactions were started by addition of GS, incubated at 22° C. for 105 min, and terminated by an addition of 10 μl of 6 N NaOH. The glucans produced were solubilized in a water bath at 80° C. for 30 min followed by an addition of 20 μl of a 4:1 diluted Sirofluor, a chemically defined fluorochrome from aniline blue. The mixtures were further incubated for 50 min at 22° C., and measured with a fluorescence spectrophotometer (FluoroLog, Horiba Jobin Yvon, Edison, N.J.) at an excitation wavelength of 390 nm and an emission wavelength of 455 nm. Standard curves were constructed using various concentrations of yeast glucan, dissolved in 300 μl of 1 N NaOH by heating 30 min at 80° C., containing the same components as the reaction mixtures except for enzyme.

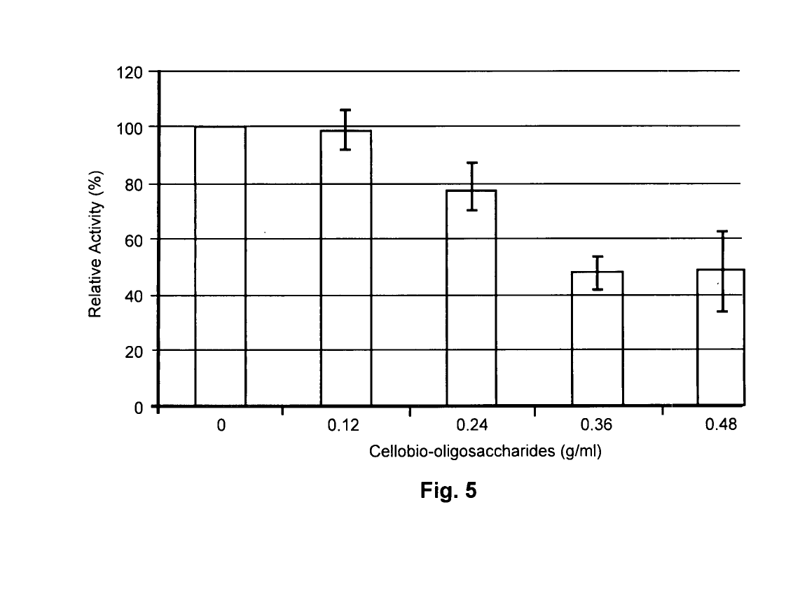

The effect of CBO on β-(1,3)-glucan synthase, the essential enzyme that forms β-(1,3)-glucan fibrils from UDP-glucose, was evaluated. Inhibition was largely dose dependent, as shown in FIG. 5. In FIG. 5, Relative Activity is the percent of 1,3-β-_{D}-glucan synthase activity (GS) at a test concentration of cellobio-oligosaccharides (CBO) as compared to the GS activity in the control (no CBO). For the determination of GS activity, 0.7 mM UDP-G was reacted with 0.83 μg/μl GS in 27 mM HEPES (pH 7.2) containing 7 μM GTP, 1.3 mM EDTA, 0.17% Brij 35, and 2.2% glycerol with the addition of 0, 0.12, 0.24, 0.36, and 0.48 g/ml CBO (primarily trisaccharides) at 22° C. for 105 min. A Sirofluor™ binding with 1,3-β-_{D}-glucans was then conducted as described above. The fluorescence was measured at an excitation wavelength of 390 nm, and an emission wavelength of 455 nm. In FIG. 5, the standard error of the mean is shown by bars. Only the lowest concentration of 0.12 g/ml CBO was insufficient to inhibit GS activity. The 50% inhibitory concentration (IC₅₀) for CBO was about 0.36 g/ml. The addition CBO above this concentration did not further decrease glucan synthase activity.

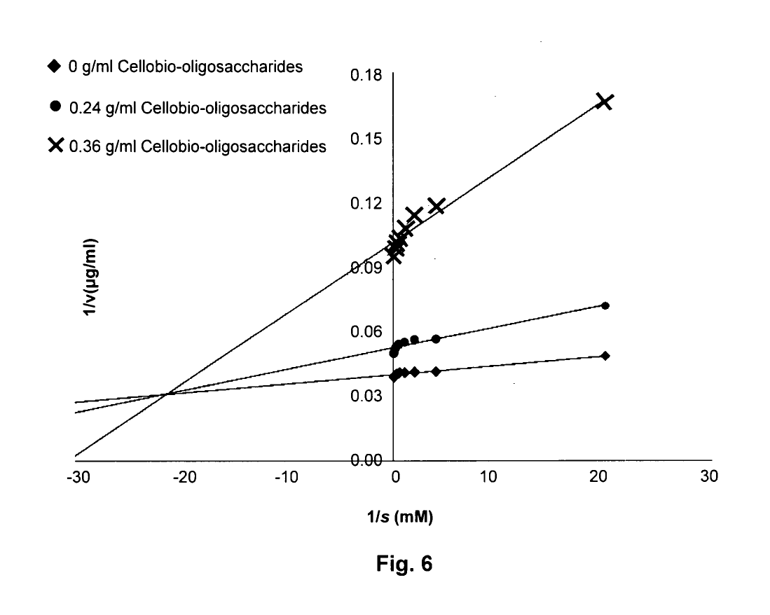

The role of CBO on glucan synthase was further evaluated by a kinetic study over a range of concentrations (0.05 to 8 mM) of UDP-glucose with CBO added at concentrations of 0, 0.24, and 0.36 g/ml. The assay mixtures contained 27 mM HEPES (pH 7.2), 7 μM GTP, 1.3 mM EDTA, 0.17% Brij 35, and 2.2% glycerol, varying concentration of UDP-G (0.05, 0.25, 0.5, 1.0, 2.0, 4.0, 6.0, and 8.0 μg/μl), and 0.83 μg/μl 1,3-β-_{D}-glucan synthase (GS). The reaction was allowed to react for 105 min at 22° C. GS activity was also measured with the addition of 0, 0.24, and 0.36 g/ml cellobio-oligosaccharides. A Sirofluor™ binding with 1,3-β-_{D}-glucans was then conducted as described above. The fluorescence was measured using an excitation wavelength of 390 nm and emission wavelength of 455 nm. The reaction velocity was calculated, and a Lineweaver-Burk plot of 1/[substrate] and 1/velocity at three oligosaccharide concentrations was plotted. These results are shown in FIG. 6. The non-parallel lines which converge at x<0 and y>0 are consistent with a mixed type of inhibition.

Morphological Changes in Fungi due to CBO. Morphologic changes in fungi due to the addition of CBO to growth media were monitored using scanning electron microscopy (SEM). Conidia (3.0×10⁴) were inoculated in PDB (potato dextrose broth) and incubated at 28° C. After incubation for 16 h, 50 mM CBO was added to one inoculated tube, and water added to a second tube as a control. The tubes were further incubated for two days at 28° C. For SEM, the incubated cultures were fixed with 2% glutaraldehyde in 0.1 M cacodylate buffer (pH 7.2) at 4° C. for 2 hs. After being washed with the buffer, specimens were post-fixed for 2 h with 1% osmium tetroxide in 0.1 M cacodylate buffer (pH 7.4) at 4° C. Samples were dehydrated in graded acetone, freeze-dried in t-butyl alcohol, and then sputter-coated with palladium-gold.

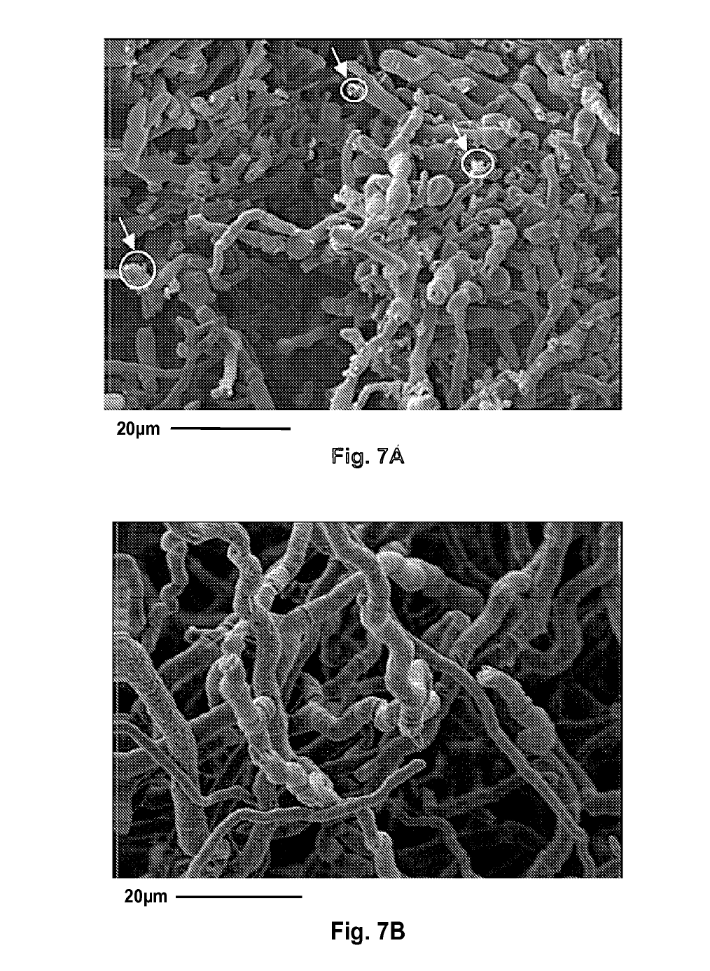

Hyphae of A. terreus showed distinct structural differences between control and CBO-treated cultures (50 mM CBO) (FIGS. 7A and 7B). The bud scar rings are found in several hyphae tips on the control (FIG. 7A) but none on CBO treated A. terreus (FIG. 7B). The hyphae of CBO-treated A. terreus fail to bud, and the population does not increase. In addition, the average width of hyphae was different between the two cultures when 20 hyphae were randomly selected and measured. The average width of twenty hyphae in CBO-treated A. terreus (3.4 μm) was 1.35 fold larger than the width in the control (2.5 μm). In the presence of CBO, the cells grew with swollen hyphae, indicating inhibition of glucan synthesis.

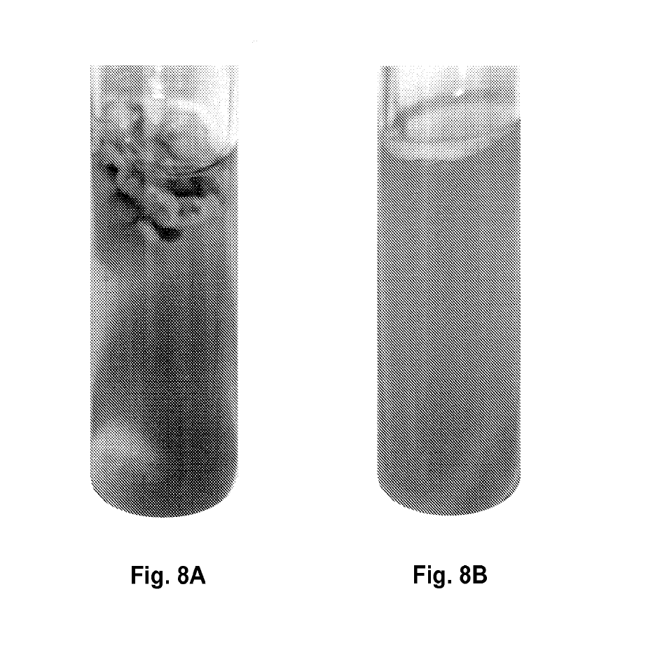

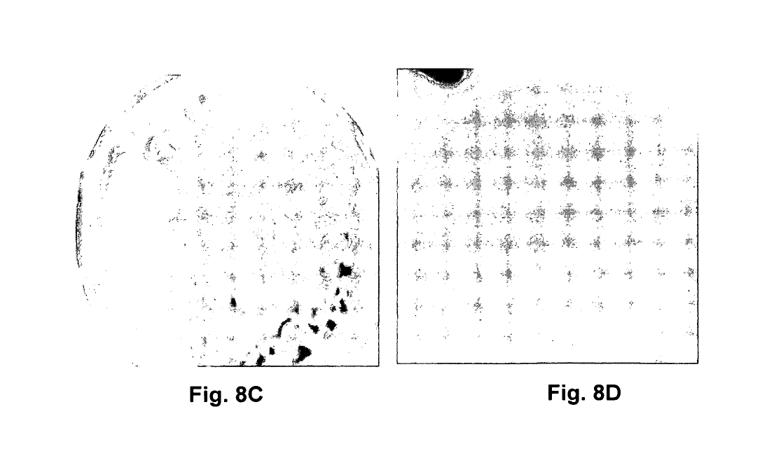

Further effects CBO on A. terreus grown in both tubes using PDB and Petri dishes using PDA were explored using an extended incubation up to ten days at 28° C. (FIG. 8). When A. terreus was grown in PDB medium, it formed tangled hyphal masses on the surface in the tube (FIG. 8A). However, these masses were not observed when A. terreus was incubated with CBO in PDB medium (FIG. 8B). There was substantial growth in the untreated culture during the course of the experiment. Similar effects were seen in the PDA petri dishes. CBO inhibited growth of A. terreus. Based on this data, CBO have great potential to function as a new antifungal agent against fungi, including the inhibition of cell wall synthesis by inhibiting 1,3-β-_{D}-glucan synthase.

The term “effective amount” as used herein refers to an amount of cellobio-oligosaccharides sufficient either to inhibit production of glucans on teeth or to inhibit the growth of fungi to a statistically significant degree (p<0.05). The term “effective amount” therefore includes, for example, an amount sufficient to reduce glucan production in the mouth or reduce fungal growth by at least 50%, and more preferably by at least 90%. The dosage ranges are those that produce the desired effect. Generally, the dosage will vary with the type of fungi, age, weight, or condition. A person of ordinary skill in the art, given the teachings of the present specification, may readily determine suitable dosage ranges. The dosage can be adjusted by the individual physician in the event of any contraindications. In any event, the effectiveness of treatment can be determined by monitoring the extent of fungal or glucan reduction by methods well known to those in the field. Moreover, the cellobio-oligosaccharides can be applied in pharmaceutically acceptable carriers known in the art. The application will be oral to reduce the glucans, and will be oral, by aspiration, or by injection for an antifungal agent. The cellobio-oligosaccharides can be a mixture of compounds with degrees of polymerization from DP 3 to 6, or can be cellobio-oligosaccharides with a single DP, preferably 3. One example of an effective mixture is one in which the majority of cellobio-oligosaccharides are trisaccharides. An example of cellobio-oligosaccharides useful as antifungal and anticariogenic agents include a mixture composed of about 66% trisaccharides with two different structures, about 13% tetrasaccharides, about 13% pentasaccharides, and about 8% hexasaccharides.

The cellobio-oligosaccharides may be administered to a patient by any suitable means, including oral, parenteral, subcutaneous, intrapulmonary, topically, and intranasal administration. Parenteral infusions include intramuscular, intravenous, intraarterial, intraperitoneal or intravitreal administration. The cellobio-oligosaccharides may also be administered orally in the form of capsules, powders, or granules.

Pharmaceutically acceptable carrier preparations for parenteral administration include sterile, aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, emulsions or suspensions, including saline and buffered media. Parenteral vehicles include sodium chloride solution, Ringer's dextrose, dextrose and sodium chloride, lactated Ringer's, or fixed oils. The cellobio-oligosaccharides may be mixed with excipients that are pharmaceutically acceptable and are compatible with the active ingredient. Suitable excipients include water, saline, dextrose, and glycerol, or combinations thereof. Intravenous vehicles include fluid and nutrient replenishers, electrolyte replenishers, such as those based on Ringer's dextrose, and the like. Preservatives and other additives may also be present such as, for example, antimicrobials, anti-oxidants, chelating agents, inert gases, and the like.

The cellobio-oligosaccharides could also be mixed with dental care products, e.g., toothpaste or mouthwash. They could also be mixed with other antifungal agents, e.g., echinocandins, pneumocandins, papulacandinsfluconazole, itraconazole, ketoconazole, miconasol, allymine, amphotericin B, nystatin, flucytosine), 5-fluorocytosine, nikkomycin, demethylallosamidin, polyxins, flocculosin, and δ-gluconolactone.

### REFERENCES

Agrawal, P. K. 1992. NMR spectroscopy in the structural elucidation of oligosaccharides and glycosides. Phy. 31: 3307-3330.
Archer, D. B. 1977. Chitin biosynthesis in protoplasts and subcellular fractions of Aspergillus fumigatus. Biochem. J. 164: 653-658.
Beauvais, A., Bruneau, J. M., Mol, P. C., Buitrago, M. J., Legrand, R., and Latge, J. P. 2001. Glucan synthase complex of Aspergillus fumigates. J. Bacteriol. 183: 2273-2279.
Bock, K., Brignole, A., and Sigurskjold, B. W. 1986. Cormormation dependence of 13C Nuclear Magnetic resonance chemical shifts in oligosaccharides. J. Chem. Soc. Perkin. Trans. II 1711-1713.
Bozzola, J. J., Mehta, R. Nisbet, L. and Valenta, J. 1984. The effect of aculeacin A and papulacandin B on morphology and cell wall ultrastructure in Candida albicans. Can. J. Microbiol. 30: 857-863.
Cassone, A., Mason, R. and Kerridge, D. 1981. Lysis of growing yeast-form cells of Candida albicans by echinocandin: a cytological study. Sabouraudia 19: 97-110.
Douglas, C. M., Foor, F., Marrinan, J. A., Morin, N., Nielsen, J. B., Dahl, A. M., Mazur, P., Baginsky, W., Li, W., el-Sherbeini, M., and et al. 1994. The Saccharomyces cerevisiae FKS1 (ETG1) gene encodes an integral membrane protein which is a subunit of 1,3-β-D-glucan synthase. Proc. Natl. Acad. Sci. USA 91: 12907-12911.
Eggleston, G. and Côte, G. L. 2003. Oligosaccharides in food and agriculture. In Oligosaccharides in Food and Agriculture (Eggleston, G., Côte, G. L. eds), p2, American Chemical Society, Danvers, Mass.
Fu, D., and Robyt, J. F. 1990. Acceptors reactions of maltodextrins with Leuconostoc mesenteroides B-512FM dextransucrase. Arch. Biochem. Biophys. 283: 379-387.
Hamada, S. and Slade, H. D. 1980. Biology, immunology, and cariogenicity of Streptococcus mutans. Microbiol. Rev. 44: 331-384.
Kamat, U., Garg, R., and Sharma, C. B. 1992. Purification to homogeneity and characterization of a 1,3-β-glucan (callose) synthase from germinating Arachis hypogaea cotyledons. Arch. Biochem. Biophys. 298: 731-739.
Kaur, R., Macleod, J., Foley, W., and Nayudu, M. 2006. Gluconic acid: an antifungal agent produced by Pseudomonas species in biological control of take-all. Phytochemistry. 67: 595-604.
Kim, D. and Kim D. 1999. Facile purification and characterization of dextransucrase from Leuconostoc mesenteroides B-512FMCM. J. Microbiol. Biotechnol. 9: 219-222.
Kono, H., Waelchli, M. R., Fujiwara, M., Erata, T., and Takai, M. 1999. Transglycosylation of cellobiose by partially purified Trichoderma viride cellulase. Carbohydr. Res. 319: 29-37.
Kulakovskaya, E. V., Golubev, V. I., and corresponding member of the Ras I. S. Kulaev. 2006. Extracellular antifungal glycolipids of Cryptococcus humicola yeasts. 410: 393-395.
Kulakovskaya, E. V., Kulakovskaya, T. V., Golubev, V. I., Shashkov, A. S., Grachev, A. A., and Nifantiev, N. E. 2007. Fungicidal activity of cellobiose lipids from culture broth of yeast Cryptococcus humicola and Pseudozyma fusiformata. Russ. J. Bioorg. Chem. 33: 155-160.
Kulakovskaya, T. V., Shashkov, A. S., Kulakovskaya, E. V., and Golubev, W. I., 2005. Ustilagic acid secretion by Pseudozyma fusiformata strains. FEMS Yeast Res. 5: 919-923.
Kurtz, M. B., Heath, I. B., Marrinan, J., Dreikorn, S., Onishi, J. and Douglas, C. 1994. Morphological effects of lipopeptides against Aspergillus fumigates correlate with activities against (1,3)-β-D-glucan synthase. Antimicrob. Agents Chemother. 38: 1480-1489.
Lee, H.-Y., Kim, M.-J., Baek, J.-S., Lee, H.-S., Cha, H.-J., Lee, S.-B., Moon, T.-W., Seo, E.-S., Kim, D., Park, C.-S., and Park, K.-H. 2003. Preparation and characterization of maltosyl-sucrose isomers produced by transglycosylation of maltogenic amylase from Bacillus stearothermophilus. J. Mol. Catal. B: Enzym. 26: 293-305.
Lopez-Romeo, E., and Ruiz-Herrera, J. 1978. Properties of beta-glucan synthase from Saccharomyces cerevisiae. Antonie Leeuwenhoek. 44: 329-339.
Marechal, L. R., Goldemberg, S. H. 1964. Uridine diphosphate glucose-beta-1,3-glucan-beta-3-glucosyltransferase from Euglena gracilis. J. Biol. Chem. 239: 3163-3167.
Mimee, B., Labbé, C., Pelletier, R., and Bélanger, R. 2005. Antifungal activity of flocculosin, a novel glycolipid isolated from Pseudozyma flocculosa. Antimicrob. Agents Chemother. 49: 1597-1599.
Morales, M. A., et al.; Novel Oligosaccharides Synthesized From Sucrose Donor And Cellobiose Acceptor By Alternansucrase; Carbohydrate Research 331 (2001) 403-411.
Morrow, D. L., and Lucas, W. J. 1987. (1→3)-β-_{D}-Glucan synthase from sugar beet. Plant Physiol. 84: 565-567.
Nam, S.-H., Ko, E.-A., Jin, X.-J., Breton, V., Abada, E., Kim, Y.-M., Kimura, A., and Kim, D. 2007. Synthesis of thermo- and acid-stable novel oligosaccharides by using dextransucrase with high concentration of sucrose. J. Appl. Glycosci. 54: 147-155.
Nisizawa, T., Takeuchi, K., and Imai, S. 1986. Difference in mode of inhibition between α-_{D}-xylosyl β-_{D}-fructoside in synthesis of glucan by Streptococcus mutans _{D}-glucosyltransferase. Carbohydr. Res. 147: 135-144.
Onishi, J., Meinz, M., Thompson, J., Curotto, J., Dreikorn, S., Rosenbach, M., Douglas, C., Flattery, A., Kong, L., Cabello, A., Vicente, F., Pelaez, F., Diez, M. T., Martin, I., Bills, G., Giacobbe, R., Dombrowski, A., Schwartz, R., Morris, S., Harris, G., Tsipouras, A., Wilson, K., and Kurtz, M. B. 2000. Discovery of novel antifungal β1,3-D-glucan synthesis inhibitors. Antimicrob. Agents Chemother. 44: 368-377.
Otaka, K.; Functional Oligosaccharide and Its New Aspect as Immune Modulation; J. Bio. Macromol., 6(1), 3-9 (2006).
Puchkov, E. O., Wiese, A., Seydel, U., and Kulakovskaya, T. V., 2001. Cytoplasmic membrane of a sensitive yeast is a primary target for Cryptococcus humicola mycocidal compound (microcin). Biochim. Biophys. Acta. 1512: 239-250.
Robyt, J. F., and Eklund, S. H. 1983. Relative, quantitative effects of acceptors in the reaction of euconostoc mesenteroides B-512F dextransucrase. Carbohydr. Res. 121: 279-286.
Robyt, J. F. 1995. Glucansucrase synthesis of polysaccharides. Adv. Carbohydr. Chem. Biochem. 51: 133-168.
Ruiz-Herrera, J. 1992. Fungal cell wall: structure, synthesis, and assembly. CRC Press, Inc. Boca Raton, Fla.
Shedletzky, E., Unger, C., and Delmer D. P. 1997. A microtiter-based fluorescence assay for (1,3)-β-glucan synthases. Anal. Biochem. 249: 88-93.
Takeda, H., and Kinosh, S. 1995. Production of fructosylxylosides by Scopulariopsis brevicaulis sp. J. Ferment. Bioeng. 79: 242-246.
Tkacz, J. S. 1992. Glucan biosynthesis in fungi and its inhibition. In: Sutcliffe J. J., Georgopapadakou, N. H. eds. Emerging targets in antibacterial and antifungal chemotherapy. New York: Chapman and Hall, 495-523.
Traxler, P., Gruner, J., and Auden, J. A. 1977. Papulacandins, a new family of antibiotics with antifungal activity. Fermentation, isolation, chemical and biological characterization of papulacandins A, B, C, D, and E. J. antibiot (Tokyo). 30:289-296.
Yoon, S.-H. and Robyt, J. F. 2002. Synthesis of acarbose analogues by transglycosylation reactions of Leuconostoc mesesnteroides B-512FMC and B-742CB dextransucrase. Carbohydr. Res. 337: 2427-2435.
Yoon, S.-H., Fulton, D. B. and Robyt, J. F. 2004. Enzymatic synthesis of two salicin analogues by reaction of salicyl alcohol with Bacillus macerans cyclomaltodextrin glucanyltransferase and Leuconostoc mesenteroides B-742CB dextransucrase. Carbohydr. Res. 339: 1517-1529.

The complete disclosures of all references cited in the specification are hereby incorporated by reference. Also, incorporated by reference is the complete disclosure of the following documents: (1) Kim, Misook, “Enzymatic Production and Biological Activities of Cellobio-oligosaccharides from Lignocellulose.” Ph.D. dissertation, Louisiana State University, filed with the Graduate School and Library on Apr. 16, 2008, but withheld from public access at the author's request; and (2) U.S. Provisional Application No. 61/046,099. In the event of an otherwise irreconcilable conflict, however, the present specification shall control.

What is claimed:

1. A method for inhibiting the growth of fungi where the fungal growth depends on the activity of glucan synthase, said method comprising treating the fungi with an effective amount of a composition comprising one or more cellobio-oligosaccharides, wherein said fungal growth is substantially less than growth of the fungi without treatment with cellobio-oligosaccharides.

2. The method of claim 1, wherein the cellobio-oligosaccharides are produced by a dextransucrase-catalyzed transglycosylation reaction of sucrose with cellobiose.

3. The method of claim 1, wherein the cellobio-oligosaccharides are produced by a dextransucrase-catalyzed transglycosylation reaction of sucrose with cellobiose.

4. The method of claim 1, wherein the cellobio-oligosaccharides have a degree of polymerization ranging from 3 to 6 glucosyl groups.

5. The method of claim 1, wherein the cellobio-oligosaccharides have a degree of polymerization of 3 glucosyl groups.

6. The method of claim 5, wherein the cellobio-oligosaccharides are selected from the group consisting of α-D-glucopyranosyl-(1→2)-β-D-glucopyranosyl-(1→4)-D-glucopyranose and α-D-glucopyranosyl-(1→6)-β-D-glucopyranosyl-(1→4)-D-glucopyranose.

7. The method of claim 1, wherein the fungi is selected from the group consisting of Candida, Aphanomyces, Paracoccidioides, Saprolegma, Aspergillus and Cordyceps.

8. The method of claim 1, wherein the fungi is Aspergillus.

9. The method of claim 1, wherein the fungi is Aspergillus terreus.

10. The method of claim 1, additionally comprising administering one or more antifungal agents that are not cellobio-oligosaccharides.

11. The method of claim 10, wherein the antifungal agents are selected from the group consisting of echinocandins, pneumocandins, papulacandinsfluconazole, itraconazole, ketoconazole, miconasol, allymine, amphotericin B, nystatin, flucytosine), 5-fluorocytosine, nikkomycin, demethylallosamidin, polyxins, flocculosin, and δ-gluconolactone.

12. A method for inhibiting the growth of fungi whose growth depends on the activity of glucan synthase in a patient, said method comprising administering to the patient an effective amount of a composition comprising one or more cellobio-oligosaccharides, wherein said fungal growth is substantially reduced.

13. The method of claim 12, wherein the cellobio-oligosaccharides are produced by a dextransucrase-catalyzed transglycosylation reaction of sucrose with cellobiose.

14. The method of claim 12, wherein the cellobio-oligosaccharides are produced by a dextransucrase-catalyzed transglycosylation reaction of sucrose with cellobiose.

15. The method of claim 12, wherein the cellobio-oligosaccharides have a degree of polymerization ranging from 3 to 6 glucosyl groups.

16. The method of claim 12, wherein the cellobio-oligosaccharides have a degree of polymerization of 3 glucosyl groups.

17. The method of claim 16, wherein the cellobio-oligosaccharides are selected from the group consisting of α-D-glucopyranosyl-(1→2)-β-D-glucopyranosyl-(1→4)-D-glucopyranose and α-D-glucopyranosyl-(1→6)-β-D-glucopyranosyl-(1→4)-D-glucopyranose.

18. The method of claim 12, wherein the fungi is selected from the group consisting of Candida, Aphanomyces, Paracoccidioides, Saprolegma, Aspergillus and Cordyceps.

19. The method of claim 12, wherein the fungi is Aspergillus.

20. The method of claim 12, wherein the fungi is Aspergillus terreus.

21. The method of claim 12, additionally comprising administering one or more antifungal agents that are not cellobio-oligosaccharides.

22. The method of claim 21, wherein the antifungal agents are selected from the group consisting of echinocandins, pneumocandins, papulacandinsfluconazole, itraconazole, ketoconazole, miconasol, allymine, amphotericin B, nystatin, flucytosine), 5-fluorocytosine, nikkomycin, demethylallosamidin, polyxins, flocculosin, and δ-gluconolactone.

23. A method to inhibit the adherence of bacteria on mammalian teeth, said method comprising orally administering to the mammal an effective amount of a composition comprising one or more cellobio-oligosaccharides, wherein the number of bacteria adhering to the teeth of the treated mammal are substantially less than the number of bacteria in a mammal without a treatment of cellobio-oligosaccharides.

24. The method of claim 22, wherein the cellobio-oligosaccharides are produced by a dextransucrase-catalyzed transglycosylation reaction of sucrose with cellobiose.

25. The method of claim 22, wherein the cellobio-oligosaccharides are produced by a dextransucrase-catalyzed transglycosylation reaction of sucrose with cellobiose.

26. The method of claim 22, wherein the cellobio-oligosaccharides have a degree of polymerization ranging from 3 to 6 glucosyl groups.

27. The method of claim 22, wherein the cellobio-oligosaccharides have a degree of polymerization of 3 glucosyl groups.

28. The method of claim 27, wherein the cellobio-oligosaccharides are selected from the group consisting of α-D-glucopyranosyl-(1→2)-β-D-glucopyranosyl-(1→4)-D-glucopyranose and α-D-glucopyranosyl-(1→6)-β-D-glucopyranosyl-(1→4)-D-glucopyranose.

29. The method of claim 22, wherein the oral bacteria is Streptococcus mutans.

30. A method to inhibit the production of insoluble glucans on mammalian teeth, said method comprising orally administering to the mammal an effective amount of a composition comprising one or more cellobio-oligosaccharides, wherein the amount of insoluble glucans adhering to the teeth of the treated mammal are substantially less than the amount of insoluble glucans in a mammal without treatment with cellobio-oligosaccharides.

31. The method of claim 30, wherein the cellobio-oligosaccharides are produced by a dextransucrase-catalyzed transglycosylation reaction of sucrose with cellobiose.

32. The method of claim 30, wherein the cellobio-oligosaccharides are produced by a dextransucrase-catalyzed transglycosylation reaction of sucrose with cellobiose.

33. The method of claim 30, wherein the cellobio-oligosaccharides have a degree of polymerization ranging from 3 to 6 glucosyl groups.

34. The method of claim 30, wherein the cellobio-oligosaccharides have a degree of polymerization of 3 glucosyl groups.

35. The method of claim 34, wherein the cellobio-oligosaccharides are selected from the group consisting of α-D-glucopyranosyl-(1→2)-β-D-glucopyranosyl-(1→4)-D-glucopyranose and α-D-glucopyranosyl-(1→6)-β-D-glucopyranosyl-(1→4)-D-glucopyranose.

36. A composition for prevention of tooth decay, wherein said composition comprises a dental care product and one or more cellobio-oligosaccharides.

37. The composition of claim 36, wherein the cellobio-oligosaccharides are produced by a dextransucrase-catalyzed transglycosylation reaction of sucrose with cellobiose.

38. The composition of claim 36, wherein the cellobio-oligosaccharides are produced by a dextransucrase-catalyzed transglycosylation reaction of sucrose with cellobiose.

39. The composition of claim 36, wherein the cellobio-oligosaccharides have a degree of polymerization ranging from 3 to 6 glucosyl groups.

40. The composition of claim 36, wherein the cellobio-oligosaccharides have a degree of polymerization of 3 glucosyl groups.

41. The composition of claim 40, wherein the cellobio-oligosaccharides are selected from the group consisting of α-D-glucopyranosyl-(1→2)-β-D-glucopyranosyl-(1→4)-D-glucopyranose and α-D-glucopyranosyl-(1→6)-β-D-glucopyranosyl-(1→4)-D-glucopyranose.

42. The composition of claim 36, wherein the dental care product is selected from the group consisting of toothpaste and mouthwash.

43. A composition for inhibiting fungal growth, wherein said composition comprises one or more cellobio-oligosaccharides and one or more antifungal agents that are not a cellobio-oligosaccharide.

44. The composition of claim 43, wherein the antifungal agents are selected from the group consisting of echinocandins, pneumocandins, papulacandinsfluconazole, itraconazole, ketoconazole, miconasol, allymine, amphotericin B, nystatin, flucytosine), 5-fluorocytosine, nikkomycin, demethylallosamidin, polyxins, flocculosin, and δ-gluconolactone.

45. The composition of claim 43, wherein the cellobio-oligosaccharides are produced by a dextransucrase-catalyzed transglycosylation reaction of sucrose with cellobiose.

46. The composition of claim 43, wherein the cellobio-oligosaccharides have a degree of polymerization ranging from 3 to 6 glucosyl groups.

47. The composition of claim 43, wherein the cellobio-oligosaccharides have a degree of polymerization of 3 glucosyl groups.

48. The composition of claim 47, wherein the cellobio-oligosaccharides are selected from the group consisting of α-D-glucopyranosyl-(1→2)-β-D-glucopyranosyl-(1→4)-D-glucopyranose and α-D-glucopyranosyl-(1→6)-β-D-glucopyranosyl-(1→4)-D-glucopyranose.
